(19) United States
(12) Patent Application Publication
(10) Pub. No.: US 2026/0249047 A1
(43) Pub. Date: Aug. 27, 2026

(54) Fluid Transfer Device And Method Of Use Thereof For Delocalized Caustic Medication Delivery

(71) Applicant: Becton, Dickinson and Company, Franklin Lakes, NJ (US)

(72) Inventors: Ajay Suryavanshi, Pune (IN); Manish Kumar, Bengaluru (IN); Dorien Gemma Butter, Millcreek, UT (US); Michael Glen Beeman, Salt Lake City, UT (US)

(21) Appl. No.: 19/062,444

(22) Filed: Feb. 25, 2025

(51) Int. Cl. A61M 25/01; A61M 25/00; A61M 25/06

(52) U.S. Cl. CPC A61M 25/0113; A61M 25/0606; A61M 2025/0004; A61M 2025/0175; A61M 2202/0007; A61M 2202/049; A61M 2205/0216; A61M 2205/103; A61M 2205/50

(57) ABSTRACT

Provided herein is a system for intravenous delivery of a caustic medication to a patient. The system includes a catheter assembly, a fluid source containing a caustic medication, and a fluid transfer device coupleable to the fluid source and to the catheter assembly. The fluid transfer device includes a flexible tube, a housing configured to movably receive the flexible tube, and an advancement member configured to move relative to the housing to move the flexible tube between a first position and a second position, with the distal end of the flexible tube transferring caustic medication to the patient when in the second position. The fluid transfer device is operable to incrementally advance the flexible tube distally out past the distal tip of the catheter by a finite distance, to reposition the distal end of the flexible tube within a vein of the patient during administering of the caustic medication.

### BACKGROUND OF THE INVENTION

### Field of the Invention

The present disclosure relates generally to fluid transfer devices for use with intravenous (IV) catheters and, more specifically, to fluid transfer devices that are configured to enable administering of caustic medications through a peripheral intravenous catheter.

### Description of Related Art

Catheters are frequently utilized to administer fluids into and out of the body. Patients in a variety of settings, including in hospitals and in home care, receive fluids, pharmaceuticals, and blood products via a vascular access device (VAD) such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC) or peripherally inserted intravenous catheter (PICC), as examples.

Recent developments in the PIVC field have led to the emergence of technologies designed to facilitate fluid transfer (e.g., blood draw and fluid infusion) using an in-dwelling PIVC and accompanying fluid transfer device. The main method by which these fluid transfer devices work is by employing an introducer for inserting a flexible tube, probe, or other instrument through the catheter lumen of the PIVC, with the introducer being attached to a catheter adapter of the PIVC that provides for insertion of the flexible tube into the PIVC. For example, the catheter adapter may include a needleless access connector thereon by which the flexible tube may be introduced to provide access to the PIVC and into the patient's vasculature. In operation of the fluid transfer device, an advancement member is actuated by an operator relative to a housing to effectuate movement of the flexible tube within the housing and advance the flexible tube out therefrom for advancement into the in-dwelling PIVC. That is, the advancement member may be moved distally by the operator to cause a corresponding movement of the flexible tube relative to the housing, such that the flexible tube may be advanced out from the housing and into the in-dwelling PIVC.

With regard to the use of a VAD for purposes of fluid infusion, it is recognized that the infusion of certain caustic medications, such as chemotherapy drugs or certain antibiotics, has typically been performed via PICCs and CVCs, rather than PIVCs. That is, it is known that infusion of such caustic medications may damage the tunica intima layer of a vein, especially when delivery of such medications is to smaller diameter peripheral veins, such that caustic medications are typically infused to larger diameter veins/arteries via the use of PICCs and CVCs. While such fluid infusion is effective, it is recognized that it would be desirable to enable the infusion of caustic medications into a PIVC via a fluid transfer device as described above, as such fluid infusions could then be performed using an indwelling PIVC without the need for additional needle sticks.

Accordingly, a need exists in the art for a fluid transfer device that provides for infusion of caustic medications into an indwelling PIVC, while reducing the harmful effects of caustic medications on vein walls when delivered at the peripheral veins. The fluid transfer device should enable variation in the location of intravenous administration of the caustic medication, with such distributed delivery lessening the risk of tissue damage to the vein.

### SUMMARY OF THE INVENTION

Provided herein is a system for intravenous delivery of a caustic medication to a patient. The system includes a catheter assembly including a catheter adapter and a catheter positionable intravenously in the patient. The system also includes a fluid source containing a caustic medication and a fluid transfer device coupleable to the fluid source and to the catheter assembly and operable to administer the caustic medication to the patient. The fluid transfer device further includes a flexible tube configured to be inserted within the catheter and in fluid communication with the fluid source, a housing having a proximal end and a distal end and defining an inner volume configured to movably receive the flexible tube, a coupling device positioned at the distal end of the housing and configured to couple the housing to the catheter assembly, and an advancement member configured to move relative to the housing to move the flexible tube between a first position, in which a distal end of the flexible tube is disposed within the housing or within the coupling device, and a second position, in which the distal end of the flexible tube is disposed within the catheter or past a distal tip of the catheter, with the flexible tube able to transfer fluid from the fluid source to the patient when in the second position. With the flexible tube at the second position, the fluid transfer device is configured to further incrementally advance the flexible tube distally out past the distal tip of the catheter by a finite distance, to reposition the distal end of the flexible tube within a vein of the patient during administering of the caustic medication.

In some embodiments, the advancement member comprises a first portion and a second portion, with the first portion movably disposed along an upper surface of the housing and the second portion movably disposed within an inner volume of the housing, with the advancement member movable distally along the housing to move the flexible tube from the first position to the second position.

In some embodiments, the fluid transfer device further comprises a linear actuator configured to move the advancement member distally along the housing, the linear actuator including a motor, a ball screw, and a coupler, with the motor operating to cause rotation of the ball screw and rotation of the ball screw, in turn, causing a linear motion of the coupler, and wherein portion of the coupler engages the first portion of the advancement member, such that the linear motion of the coupler is transferred to the advancement member.

In some embodiments, the linear actuator comprises a control module having a program stored thereon operates the motor to cause the distal end of the flexible tube to be incrementally advanced distally out past the distal tip of the catheter by a finite distance for each actuation of the motor, to reposition the distal end of the flexible tube within the vein of the patient.

In some embodiments, the housing comprises an outer housing, wherein the fluid transfer device further comprises an inner housing slidably received within the outer housing, the inner housing including a grooved track formed along a top surface thereof, with a proximal end of the flexible tube secured to the inner housing, and wherein the advancement member comprises an advancement wheel secured to the outer housing that is rotatable in a first direction and a second direction, where rotation in the second direction causes the flexible tube to move toward the second position, with the advancement wheel comprising teeth thereon that interact with the grooved track to advance the inner housing and the flexible tube distally when the advancement wheel is rotated in the second direction.

In some embodiments, rotation of the advancement wheel in the second direction causes the flexible tube to advance distally, with each engagement of the teeth of the advancement wheel and the grooved track resulting from rotation of the advancement wheel moving the flexible tube in the distal direction by a known finite increment, to reposition the distal end of the flexible tube within the vein of the patient.

In some embodiments, the fluid transfer device further comprises an actuator configured to rotate the advancement wheel in the first direction and the second direction, the actuator including a motor, a ball screw, a coupler, and a linkage arrangement, with the linkage arrangement connecting the coupler to the advancement wheel, wherein the motor operates to cause rotation of the ball screw and rotation of the ball screw, in turn, causing a linear motion of the coupler, and the linkage arrangement translating the linear motion of the coupler into a rotational motion that is applied to the advancement wheel to cause rotation thereof in the first direction or the second direction.

In some embodiments, the actuator comprises a control module having a program stored thereon operates the motor to cause the distal end of the flexible tube to be incrementally advanced distally out past the distal tip of the catheter by a finite distance for each actuation of the motor, to reposition the distal end of the flexible tube within the vein of the patient.

In some embodiments, the flexible tube comprises a telescoping flexible tube comprising a main flexible tube and an inner telescoping tube portion positioned at a distal end of the main flexible tube, the inner telescoping tube portion movable between an initial position where the inner telescoping tube portion is nested substantially or completely within the main flexible tube and an extended position where the inner telescoping tube portion extends out further past the distal end of the main flexible tube.

In some embodiments, the main flexible tube comprises a first lip formed on an inner surface thereof and the inner telescoping tube portion comprises a second lip formed on an outer surface thereof, and wherein the inner telescoping tube portion is retained in the initial position when the first lip and the second lip are aligned and engaged and the inner telescoping tube portion is released from the main flexible tube when the first lip and the second lip are misaligned and disengaged, thereby allowing the inner telescoping tube portion to move to the extended position.

In some embodiments, the first lip and the second lip are moved from an aligned and engaged arrangement to a misaligned and disengaged arrangement responsive to rotation of the housing of the fluid transfer device.

In some embodiments, with the inner telescoping tube portion released from the main flexible tube, the inner telescoping tube portion moves from the initial position to the extended position responsive to friction forces exerted on the inner telescoping tube portion by a flow of the caustic medication as it is administered to the patient.

In some embodiments, the telescoping flexible tube further comprises an adhesive applied between an inner surface of the main flexible tube and an outer surface of the inner telescoping tube portion, the adhesive retaining the inner telescoping tube portion in the initial position.

In some embodiments, the adhesive is configured to dissolve upon contact with the caustic medication that is infused to the patient, to release the inner telescoping tube portion from the main flexible tube, thereby allowing the inner telescoping tube portion to move to the extended position, wherein with the inner telescoping tube portion released from the main flexible tube, the inner telescoping tube portion moves from the initial position to the extended position responsive to friction forces exerted on the inner telescoping tube portion by a flow of the caustic medication as it is administered to the patient.

In some embodiments, the fluid transfer device further comprises a translating mechanism including an actuator button that extends up through an outer surface of the housing at the distal end thereof, a chuck ring positioned within the housing and/or the coupling device and about the flexible tube, a clamping chuck positioned within the coupling device and movable relative to the chuck ring, with the clamping chuck operably connected to the actuator button, and a spring positioned about a portion of the clamping chuck and within the housing, wherein depression of the actuator button transfers an actuating force and motion to the clamping chuck that causes the clamping chuck to distally advance relative to the chuck ring and to pinch down onto the flexible tube, such that the flexible tube is gripped thereby, with the distal advancing of the clamping chuck causing a corresponding incremental distal movement of the flexible tube by a finite distance, to reposition the distal end of the flexible tube within the vein of the patient.

In some embodiments, the clamping chuck moves distally when the actuator is depressed until reaching a proximal end of a cavity within the coupling device, and wherein the clamping chuck releases the flexible tube and moves back proximally within the coupling device upon release of the actuator button, with a biasing force of the spring causing the proximal movement of the clamping chuck back to an initial position.

In some embodiments, the translating mechanism further comprises a return spring positioned within the housing and configured to retract the flexible tube upon the flexible tube reaching a full distal position, with the return spring compressed by the advancement member when the advancement member is moved to a distal-most position on the housing and applying a proximally-directed force to the advancement member that causes the advancement member and the flexible tube to retract back proximally.

Also provided herein is a method of intravenously delivering a caustic medication to a patient. The method includes connecting the fluid transfer device to the catheter assembly with the distal tip of the indwelling intravenously in the patient, connecting the fluid source to the fluid transfer device such that the fluid source is fluidly connected with the flexible tube, actuating the advancement member to move the flexible tube from the first position to the second position, and with the flexible tube in the second position, operating the fluid source to administer the caustic medication to the patient, via the flexible tube. During administration of the caustic medication to the patient, the fluid transfer device is operated to move the distal end of the flexible tube to a plurality of positions that are all distal to the distal tip of the catheter, to vary an intravenous location within the patient at which the caustic medication is administered.

In some embodiments, operating the fluid transfer device during administration of the caustic medication to move the distal end of the flexible tube comprises operating a translating mechanism of the fluid transfer device to move the distal end of the flexible tube in a semi-automatic manner.

In some embodiments, operating the fluid transfer device during administration of the caustic medication to move the distal end of the flexible tube comprises extending a nested inner telescoping tube portion out from the flexible tube, to reposition a distal-most tip of the inner telescoping tube portion intravenously within the patient.

### BRIEF DESCRIPTION OF THE DRAWINGS

FIG. 1 is a perspective view of a catheter system, according to an aspect or embodiment of the present application;

FIG. 2 is a perspective view of a fluid transfer device of the system of FIG. 1, according to an aspect or embodiment of the present application;

FIG. 3 is an exploded view of the fluid transfer device of FIG. 2;

FIG. 4 is a side cross-sectional view of the fluid transfer device of FIG. 2, showing a flexible tube thereof in a first, retracted position;

FIG. 5 is a side cross-sectional view of the fluid transfer device of FIG. 2, showing the flexible tube in a second, extended position;

FIG. 6 is a schematic illustration showing positioning of a flexible tube of the fluid transfer device of FIG. 2 at a number of different intravenous locations, according to an aspect or embodiment of the present application;

FIG. 7 is a top view illustrating the fluid transfer device of FIG. 2 with a linear actuator provided to move the advancement member thereof, according to an aspect or embodiment of the present application;

FIG. 8 is a side schematic view of the fluid transfer device of FIG. 7 illustrating the linear actuator moving the flexible tube to a first position within the vein of a patient;

FIG. 9 is a side schematic view of the fluid transfer device of FIG. 7 illustrating the linear actuator moving the flexible tube to a second position within the vein of a patient;

FIG. 10 is a side view illustrating the fluid transfer device of FIG. 2 with a translating mechanism provided to advance the flexible tube thereof, according to an aspect or embodiment of the present application;

FIG. 11 is a cross-sectional view of a portion of the fluid transfer device of FIG. 10 taken along line 10-10, showing a clamping chuck and the flexible tube in a first position;

FIG. 12 is a cross-sectional view of a portion of the fluid transfer device of FIG. 10 taken along line 10-10, showing the clamping chuck and the flexible tube in a second position;

FIG. 13 is a cross-sectional view of a portion of the fluid transfer device of FIG. 10 taken along line 10-10, showing the clamping chuck in the first position and the flexible tube in the second position;

FIG. 14A is a side cross-sectional view of a portion of a telescoping flexible tube that may be included in the fluid transfer device of FIG. 2, according to an aspect or embodiment of the present application, with an inner telescoping tube portion in an initial position;

FIG. 14B is an end cross-sectional view of the telescoping flexible tube of FIG. 14A;

FIG. 15A shows the telescoping flexible tube of FIG. 14A, with the inner telescoping tube portion in an extended position;

FIG. 15B is an end cross-sectional view of the telescoping flexible tube of FIG. 15A;

FIG. 16 is a side cross-sectional view of a portion of a telescoping flexible tube that may be included in the fluid transfer device of FIG. 2, according to an aspect or embodiment of the present application, with an inner telescoping tube portion in an initial position;

FIG. 17 shows the telescoping flexible tube of FIG. 16, with the inner telescoping tube portion in an extended position;

FIG. 18 is a perspective view of a fluid transfer device, according to another aspect or embodiment of the present application;

FIG. 19 is a cross-sectional view of a portion of the fluid transfer device of FIG. 18 taken along line 18-18;

FIG. 20 is a top view illustrating the fluid transfer device of FIG. 18 with an actuator provided to rotate the advancement member thereof, according to an aspect or embodiment of the present application;

FIG. 21 is a cross-sectional view of a portion of the fluid transfer device of FIG. 18 taken along line 18-18, showing the actuator operating to advance the flexible tube to a first position within the vein of a patient; and

FIG. 22 is a cross-sectional view of a portion of the fluid transfer device of FIG. 18 taken along line 18-18, showing the actuator operating to advance the flexible tube to a second position within the vein of a patient.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward the distal tip of the needle or catheter of the system, and the terms “in the proximal direction” and “proximally” mean in the direction opposite the direction of the distal tip of the needle or catheter.

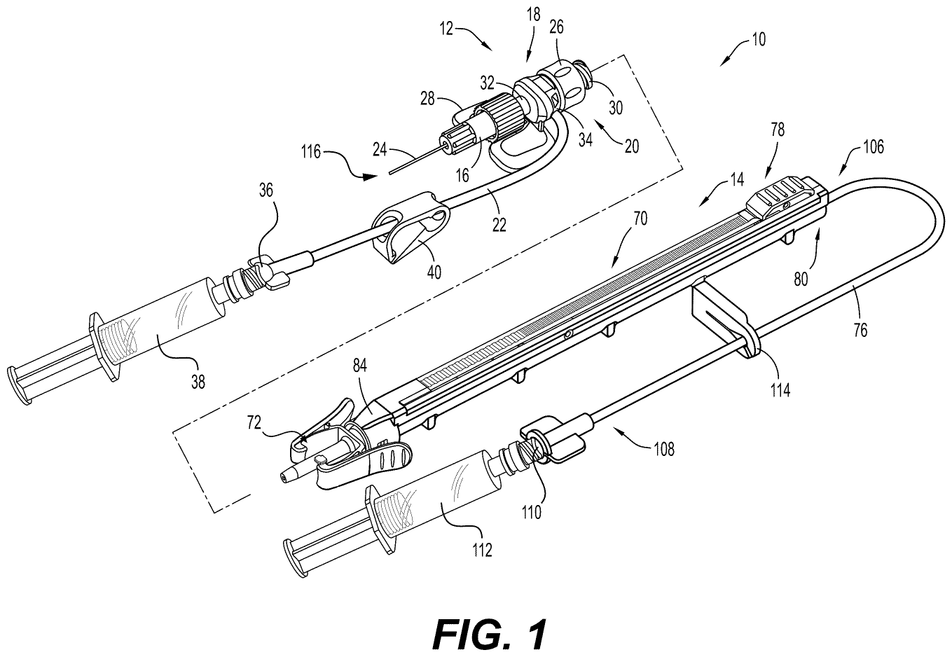

Referring to FIG. 1, shown is a non-limiting embodiment of a catheter system 10 for facilitating infusion of a caustic medication (e.g., chemotherapy drugs or certain antibiotics, as non-limiting examples) to a patient, according to one aspect or embodiment with which embodiments of the invention may be implemented. The catheter system 10 includes a catheter assembly 12 with which an associated fluid transfer device 14 may be used.

In accordance with one embodiment, the catheter assembly 12 includes a catheter hub 16 and a non-integrated extension set 18 that includes a catheter stabilizing catheter connector 20 (hereafter “catheter connector 20”), along with extension tubing 22. The catheter hub 16 receives a catheter 24 and is coupled to the catheter connector 20. In one aspect or embodiment, the catheter hub 16 may be an AccuCath™ catheter system commercially available from Becton, Dickinson and Company.

The catheter connector 20 is configured to be placed in contact with the skin surface of a patient at or near an insertion site of catheter 24. The catheter connector 20 can be any suitable shape, size, and/or configuration. In the illustrated non-limiting embodiment, the catheter connector 20 has a connector portion 26 and a stabilization portion 28. The connector portion 26 has a proximal port with a proximal coupler 30, a distal port with a distal coupler 32, and defines at least one lumen (not shown) extending through or otherwise in fluid communication with the couplers 30, 32. The proximal coupler 30 and/or the distal coupler 32 can be, for example, male or female luer locks and/or any other suitable coupler. In the illustrated embodiment, the proximal coupler 30 is provided as a needleless access connector (i.e., needle-free connector (NFC)) hereafter “needleless access connector 30”—that can be physically and fluidically coupled to peripheral devices such as fluid transfer device 14, which may comprise a PIVO™ device from Becton, Dickinson and Company, as a non-limiting example. The proximal coupler 30 presents a female luer connection/lock to which fluid transfer device 14 may be connected. The distal coupler 32 can be physically and fluidically coupled to catheter hub 16 such that the lumen of the connector portion 26 is at least selectively in fluid communication with the catheter 24. In some embodiments, an inner surface of the connector portion 26 can be configured to provide alignment, guidance, centering, etc., to an object or device (e.g., a blood draw catheter or the like, as described above) being advanced therethrough.

The connector portion 26 may also include and/or define one or more additional ports, such as a side port 34. Side port 34 can be included in and/or can be a part of the connector portion 26, the proximal coupler 30, the distal coupler 32, and/or a combination thereof. In some instances, positioning side port 34 in a desired position along a length of the connector portion 26, between the couplers 30, 32 can allow for a reduced length of the connector portion 26 and/or can facilitate flushing and/or fluid transfer via side port 34.

Side port 34 defines a lumen (not shown) that is in fluid communication with the lumen between distal coupler 32 and proximal coupler 30. In other words, the connector portion 26 and/or the side port 34 can include and/or define a first lumen (e.g., between distal coupler 32 and proximal coupler 30) and a second lumen (e.g., lumen defined by side port 34 and extension tubing 22). As such, side port 34 can provide access to the lumen between distal coupler 32 and proximal coupler 30, which in turn can provide access to a device (e.g., a VAD) that is coupled to the distal coupler 32 and/or can provide access to a portion of the body in which the VAD is at least partially disposed.

In some embodiments, the arrangement of side port 34 can be such that the connector portion 26 forms, for example, a Y-connector or a T-connector. More particularly, side port 34 can be disposed substantially perpendicular (e.g., about 90 degrees) to the lumen of the connector portion 26 and near or adjacent the proximal coupler 30. In non-limiting embodiments, side port 34 extends from connector portion 26 at an angle, relative to the lumen between distal coupler 32 and proximal coupler 30, that is not 90 degrees (e.g., side port 34 extends at an angle of, for example and without limitation, 15-165 degrees, with all values and subranges therebetween inclusive). In non-limiting embodiments, more than one side port 34 is included in connector portion 26.

As shown in FIG. 1, the side port 34 of the catheter connector 20 may be coupled to a distal end of extension tubing 22, such that the extension tubing 22 is in fluid communication with the lumen of the side port 34. A proximal end portion of the extension tubing 22 may comprise a proximal access port 36, which may be configured as a luer connection. In some embodiments, proximal access port 36 may include a needleless access connector provided thereon. According to aspects of the disclosure, the side port 34 and the extension tubing 22 can be and/or can form at least a portion of a fluid line that can be used to deliver fluid to the catheter assembly 12. For example, a (second) fluid source 38, such as a pre-filled syringe, may be fluidly connected to side port 34 and extension tubing 22 via proximal access port 36, to deliver fluid intravenously to the patient via catheter 24. In some embodiments, the fluid source 38 may deliver a non-caustic medication or saline solution to catheter assembly 12 that dilutes the caustic medication infused from the fluid source 112. In some embodiments, a clamp 40 may be provided on the extension tubing 22, with the clamp 40 configured to selectively restrict flow through the extension tubing 22.

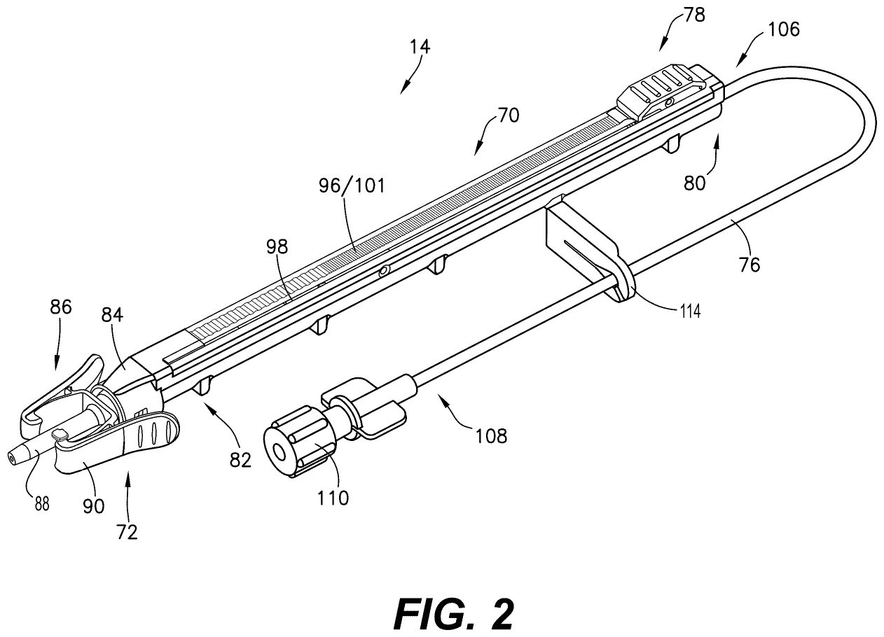

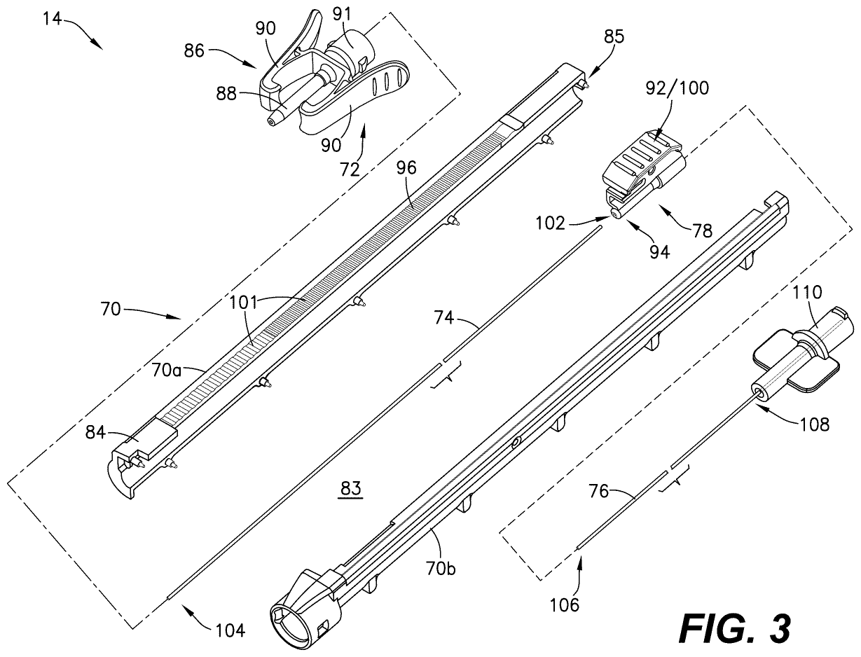

Referring still to FIG. 1 and now also to FIGS. 2 and 3, the fluid transfer device 14 (hereafter “fluid transfer device 14”) includes a housing 70, a coupling device 72, a flexible tube 74, a secondary catheter 76, and an advancement member 78. As described in further detail herein, a user can transition the fluid transfer device 14 from a first configuration to a second configuration to advance the flexible tube 74 through an existing, placed, and/or indwelling catheter 24 (i.e., when the fluid transfer device 14 is coupled thereto) such that at least an end portion of the flexible tube 74 is disposed in a distal position relative to the indwelling catheter 24. Moreover, with indwelling catheters each having a shape, size, and/or configuration that can vary based on, for example, a manufacturer of the indwelling catheter 24 and/or its intended usage, the fluid transfer device 14 can be arranged to be coupled to an indwelling catheter 24 having any suitable configuration and subsequently, to advance at least a portion of a tube through the indwelling catheter 24. In addition, the fluid transfer device 14 can be manipulated by a user to place a distal surface of the flexible tube 74 a predetermined and/or desired distance beyond a distal surface of the indwelling catheter 24 to be disposed within a portion of a vein that is targeted to receive an administered fluid.

The housing 70 of fluid transfer device 14 can be an elongate member having a proximal end 80 and a distal end 82 and defining an inner volume 83. In some embodiments, the housing 70 may be formed of a pair of housing portions 70a, 70b that are coupled together to define the inner volume 83. The housing 70 may include one or more features or surface finishes on an outer surface thereof that can be arranged to increase the ergonomics of the fluid transfer device 14, which in some instances can allow a user to manipulate the fluid transfer device 14 with one hand (i.e., single-handed use). The distal end 82 of the housing 70 can include a coupler 84 formed thereon configured to mate with the coupling device 72 of fluid transfer device and, in one embodiment, the coupler 84 may include a threaded inner surface that engages the coupling device 72. Additionally, the proximal end 80 of the housing 70 can include an opening or port 85 configured to receive a portion of the secondary catheter 76 (i.e., the portion of the secondary catheter 76 extending proximally out from housing 70) and provide for advancement and retraction of the secondary catheter 76 through the port 85.

The coupling device 72 of fluid transfer device 14 is provided at the distal end 82 of the housing 70, with the coupling device 72 providing for reversible coupling of the fluid transfer device 14 to catheter assembly 12, such as via proximal coupler 30 as shown in FIG. 1. As previously described, proximal coupler 30 may be provided as a needleless access connector and is thus referred to hereafter as “needleless access connector 30.” In some embodiments, the coupling device 72 is configured as a lock 86 that includes a blunted cannula 88 and locking arms 90 for coupling to the needleless access connector 30 of catheter assembly 12, with the blunted cannula 88 and locking arms 90 forming three points of contact therewith. However, those of skill will appreciate that any connection or coupling, for example a luer, can be used, so long as a distal end of flexible tube 74 may pass through the coupling device 72 to access catheter assembly 12. Additionally, a proximal end of the coupling device 72 may be configured as a threaded connection 91 (i.e., a threaded outer surface) that is configured to engage the coupler 84 of housing 70—with the threaded connection 91of coupling device 72 engaging the threaded inner surface of coupler 84.

In accordance with some aspects of the disclosure, the advancement member 78 of fluid transfer device 14 includes a first portion 92 and a second portion 94. The first portion 92 is movably disposed along an upper surface 96 of the housing 70 and the second portion 94 is movably disposed within the inner volume 83 of the housing 70. The arrangement of the advancement member 78 and the housing 70 is such that a connecting portion (not shown) of the advancement member 78 that joins the first and second portions 92, 94 is seated within a slot 98 formed in the upper surface 96 of the housing 70—the slot 98 generally extending between the proximal and distal ends 80, 82 of the housing 70. As the first and second portions 92, 94 are joined together, movement of the first portion 92 along the upper surface 96 of the housing 70 results in a corresponding movement of the second portion 94 within the inner volume 83.

As shown in FIGS. 2 and 3, the first portion 92 of the advancement member 78 may be configured as a tab 100 having a contact surface engageable by a user and an underside that is in contact with the outer surface 96 of the housing 70. In such embodiments, the upper surface 96 of the housing 70 can include a track 101, for example, a set of ribs, ridges, bumps, grooves, and/or the like along which the underside of tab or protrusion advances when the advancement member 78 is engaged by a user. In this manner, a user can engage the first portion 92 of the advancement member 78 and can move the advancement member 78 relative to the housing 70.

As further shown in FIG. 3, the second portion 94 includes an opening 102 extending therethrough that is configured to grip or retain a portion of the flexible tube 74. Due to a portion of the flexible tube 74 being retained within the opening 102 of second portion 94, movement of the advancement member 78 relative to housing 70 causes a corresponding movement of the flexible tube 74 relative to the housing 70. In this manner, a distal end 104 of the flexible tube 74 can be selectively moved out of or back into the inner volume 83 of the housing 70 as desired, such as advancing the distal end 104 of the flexible tube 74 out of the housing 70 when the fluid transfer device 14 has been coupled to the catheter assembly 12 and a fluid infusion/delivery procedure is to be performed.

In addition to receiving flexible tube 74 therein, the second portion 94 also grips or retains a portion of secondary catheter 76 therein. That is a distal end 106 of secondary catheter 76 is secured to second portion 94 (i.e., in opening 102 thereof) and extends proximally out therefrom, with the secondary catheter 76 extending out through opening 85 of the housing 70. A proximal end 108 of the secondary catheter 76 may include a coupler 110 thereon that may be coupled to a (first) fluid source 112 (FIG. 1), such as a fluid reservoir or pre-filled syringe, with the secondary catheter 76 establishing fluid communication between the fluid source 112 and the flexible tube 74. Additionally, in some embodiments, the secondary catheter 76 may also have a clamp 114 provided thereon, such as a slide clamp or pinch clamp, to facilitate stoppage of flow through the secondary catheter 76.

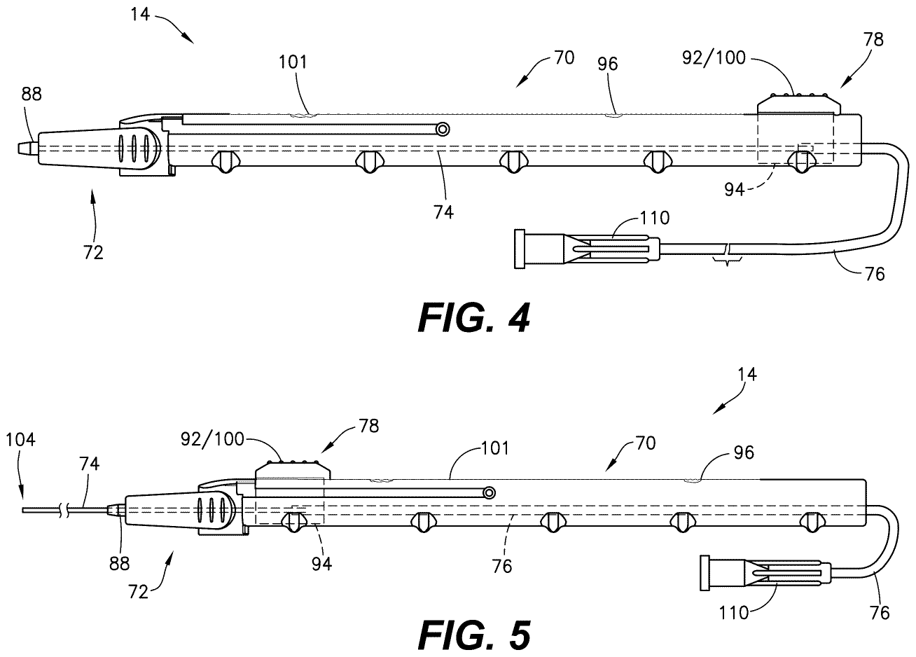

Referring now to FIGS. 4 and 5, changing of the fluid transfer device 14 between the first configuration and second configuration is shown. The fluid transfer device 14 can be in the first configuration prior to use and can be transitioned by a user (e.g., a doctor, physician, nurse, technician, phlebotomist, and/or the like) from the first configuration (FIG. 4) to the second configuration (FIG. 5) to dispose at least the distal end 104 of the flexible tube 74 in a distal position relative to the housing 70.

The fluid transfer device 14 is in the first configuration of FIG. 4 when the flexible tube 74 is disposed in the first position within the housing 70. In some embodiments, substantially the entire flexible tube 74 is disposed within the housing 70 when the flexible tube 74 is in the first position. In other embodiments, the flexible tube 74 is disposed within the housing 70 and the lock 86 when flexible tube 74 is in the first position.

The advancement member 78 is disposed in a proximal position when the fluid transfer device 14 is in the first configuration, and the user may engage the tab 100 of advancement member 78 to move the advancement member 78 relative to the housing 70, which in turn, moves the flexible tube 74 from the first position (e.g., disposed within the housing 70) toward the second position. In this manner, the flexible tube 74 is moved through the inner volume 83 of housing 70 and through the lock 86 and, as such, at least the distal end 104 of the flexible tube 74 is disposed outside of and distal to the lock 86.

The fluid transfer device 14 is in the second configuration of FIG. 5 when the flexible tube 74 is disposed in the second position. The second position of the flexible tube 74 is reached when the distal end 104 of the flexible tube 74 is placed in a desired position relative to a distal tip of the catheter 24. In some instances, for example, a distal end 104 of the flexible tube 74 can be substantially flush with a distal tip of the catheter 24 when the flexible tube 74 is in the second position. In other instances, the distal end 104 of the flexible tube 74 can extend a predetermined distance beyond the distal tip of the catheter 24, such that the distal end 104 of the flexible tube 74 is positioned within the vein at a predetermined distance beyond the distal end of the flexible tube 74.

With the flexible tube 74 in the second position (e.g., with the fluid transfer device 14 in the second configuration shown, for example, in FIG. 5), the user can establish fluid communication between the fluid source 112 and the flexible tube 74. The fluid transfer device 14 can then deliver/infuse a volume of fluid from fluid source 112 into the vein of the patient based at least in part on disposing the distal end 104 of the flexible tube 74 at the predetermined and/or desired distance beyond the distal surface of the catheter 24 (FIG. 1). In some instances, once a desired volume of a drug has been delivered to the patient, the user can move the advancement member 78 in the proximal direction, such as moving the advancement member 78 back to its proximal most position, so as to move the flexible tube 74 back to the first position.

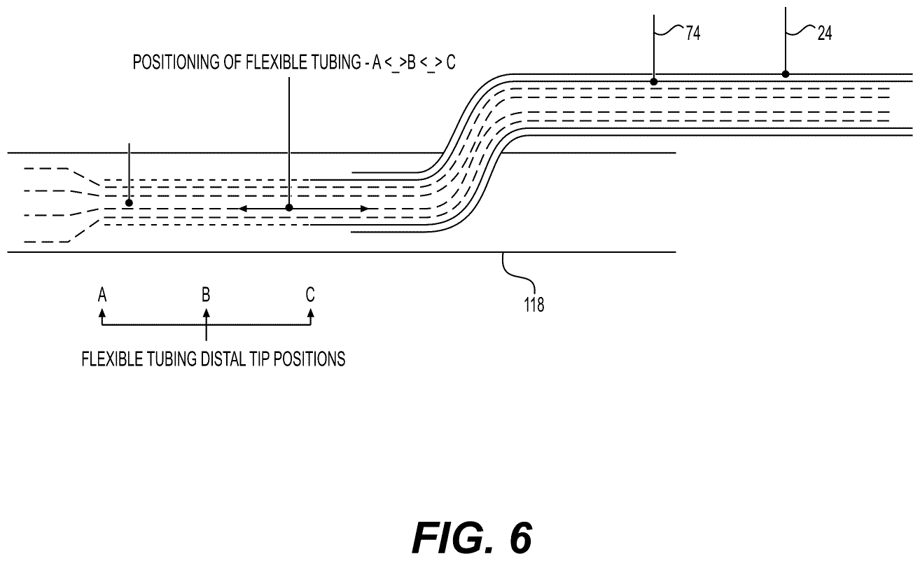

According to aspects of the disclosure, one potential use of the system 10 is for the infusion or administering of a caustic medication, such as a chemotherapy drug or antibiotic. When infusing a caustic medication, it is known that infusion of such a caustic medication may damage the tunica intima layer of a vein, especially when delivery of such medication is to smaller diameter peripheral veins. Accordingly, fluid transfer device 14 may be configured to provide for infusion of a caustic medication, while limiting/reducing the harmful effects of caustic medications on vein walls. Specifically, the fluid transfer device 14 may be configured and/or controlled during operation, either manually or semi-automatically (e.g., using an externally-controlled motorized mechanism), to vary/adjust the positioning of the distal end 104 of flexible tube 74 during infusion of the caustic medication, in order to optimize delivery of the caustic medication and maintain vein health. That is, the flexible tube 74 may be pulled/pushed relative to the catheter 24 (and within the vein) throughout the duration of a caustic medication infusion/therapy, such that the distal end 104 of the flexible tube 74 may be displaced/shifted within the vein and the administering of the caustic medication may be de-localized, which lessens the risk of tissue damage in the tunica intima layer. For example, and as shown in FIG. 6, the location of the distal end 104 of the flexible tube 74 may be shifted between a Position A, Position B, and Position C during administering of the caustic medication via actuation of the fluid transfer device 14, to de-localize delivery of the caustic medication and maintain vein health. Hence, faster healing of the vein (i.e., of the tunica intima) may be attained compared to infusion of the caustic medication at only a single location within the vein.

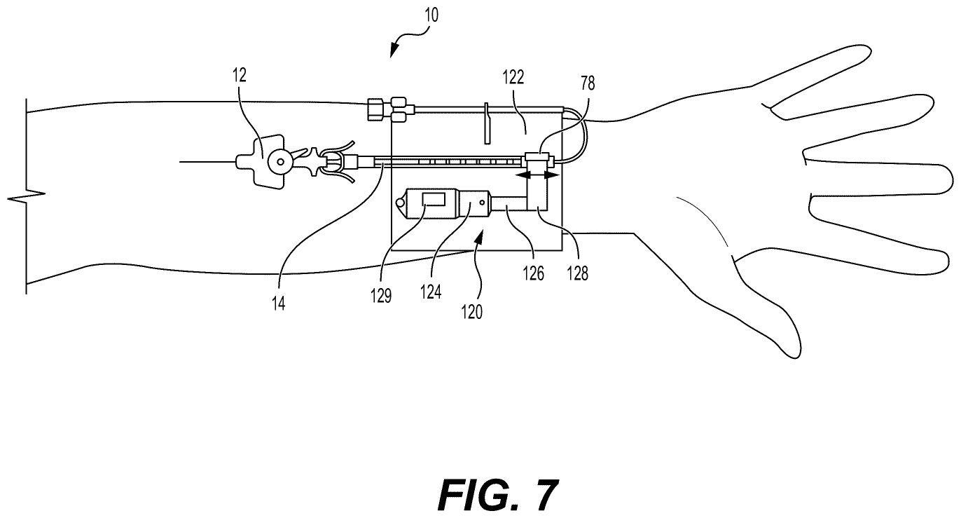

According to one aspect of the disclosure, and as shown in FIGS. 7-9, the fluid transfer device 14 may be further configured to include an externally-controlled, motorized translating mechanism 120 (hereafter, “translating mechanism”) that provides for semi-automatic control in adjusting the positioning of the distal end 104 of flexible tube 74 during infusion of the caustic medication. The translating mechanism 120 may be secured to a patient (alone or along with fluid transfer device 14) at a location adjacent to an insertion site of the catheter 24, such as via a stabilization device 122 secured to the skin of the patient. As explained in further detail below, the translating mechanism 120 is further attached/coupled to the fluid transfer device 14, i.e., to advancement member 78, such that the translating mechanism 120 may operate to cause a semi-automatic movement of the flexible tube 74 during infusion of the caustic medication.

According to one embodiment, and as shown in FIGS. 7-9, the translating mechanism 120 may be configured as a linear actuator that generally includes a motor 124, a ball screw 126, and a nut or coupler 128. The motor 124 is operable to cause rotation of the ball screw 126, with rotation of the ball screw 126 in turn causing a linear motion of the coupler 128 (which is positioned about the ball screw 126). A portion of the coupler 128 extends out to engage the advancement member 78 of the fluid transfer device (e.g. being snapped onto advancement member 78), such that linear motion of the coupler 128 caused by rotation of the motor 124 and ball screw 126 is transferred to the advancement member 78—thereby causing the advancement member 78 to translate along the track 101 of housing 70, which also moves the flexible tube 74. In some embodiments, translating mechanism 120 may control movement of the advancement member 78 to provide for a full range of positioning of the flexible tube 74 between the first position and the second position thereof, as previously described. In other embodiments, the translating mechanism 120 may control movement of the advancement member 78 to provide for precise positioning of the flexible tube 74 as it approaches the second position-in which case the translating mechanism 120 would be provided and attached to the fluid transfer device 14 after an initial actuation of the advancement member 78 from the first position. In either case, the translating mechanism 120 is operable to precisely control positioning of the distal end 104 of flexible tube 74 relative to the distal tip 116 of the catheter 24 during infusion of the caustic medication, in order to delocalize delivery of the caustic medication inside the vein 118.

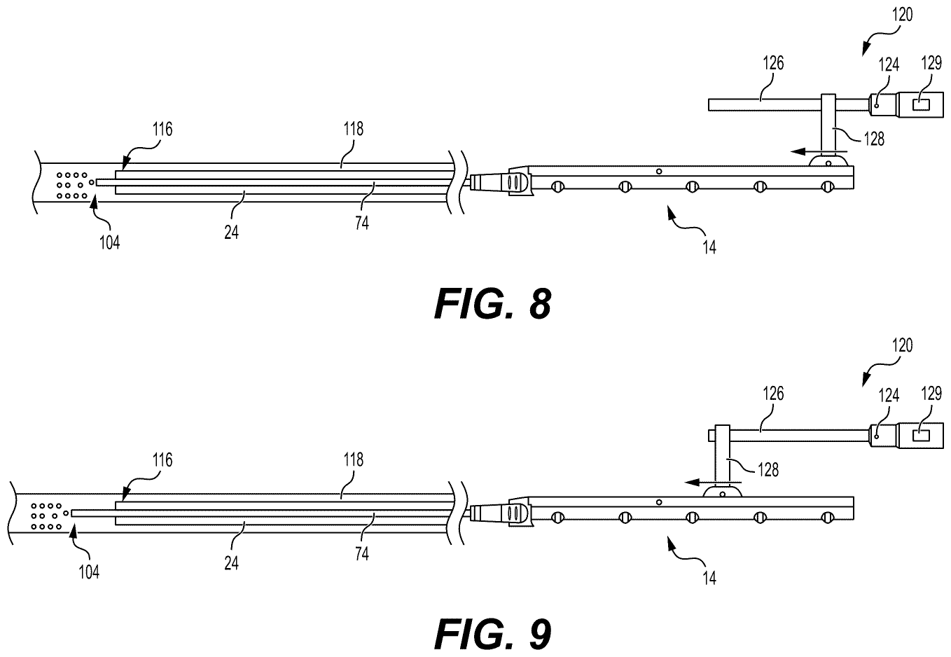

Operation of the translating mechanism 120 to cause movement of the advancement member 78 and flexible tube 74 is shown in FIGS. 8 and 9. As shown first in FIG. 8, translating mechanism 120 may be controlled to perform a first actuation, where motor 124 causes ball screw 126 to rotate and cause a corresponding linear movement of coupler 128—with the coupler 128 transferring this linear movement to advancement member 78. As the advancement member 78 is advanced distally along housing 70, the flexible tube 74 also is moved distally within catheter 24—with FIG. 8 showing movement of the flexible tube 74 that repositions the distal end 104 from a location even with the distal tip 116 of catheter 24 (or out slightly distal to the distal tip 116) to a first location where the distal end 104 extends distally past the distal tip 116 by a first amount/distance. Movement of the distal end 104 flexible tube 74 to the first position may be performed while infusion of the caustic fluid is occurring.

As shown first in FIG. 9, translating mechanism 120 may be controlled to perform a second actuation, where motor 124 causes ball screw 126 to rotate and cause a corresponding linear movement of coupler 128—with the coupler 128 transferring this linear movement to advancement member 78. As the advancement member 78 is advanced distally along housing 70, the flexible tube 74 also is moved distally within catheter 24—with FIG. 9 showing movement of the flexible tube 74 that repositions the distal end 104 from a first location that is distally past the distal tip 116 by a first amount/distance to a second location where the distal end 104 extends distally past the distal tip 116 by a second amount/distance. Again, movement of the distal end 104 flexible tube 74 to the second position may be performed while infusion of the caustic fluid is occurring. In this manner, administering of the caustic medication is de-localized, which lessens the risk of tissue damage in the vein 118.

According to embodiments, the translating mechanism 120 may comprise a battery-operated, re-usable device, that may be selectively attached and detached from the fluid transfer device 14. The motor 124 of translating mechanism 120 may be controlled according to a set program, which may be stored on a control module 129 associated with the motor 124, with the program configured to operate the motor 124 to cause the distal end 104 of the flexible tube 74 to be moved intravenously to a number of pre-determined locations within the vein-i.e., moved distally out past the distal tip 116 of the catheter 24 by a number of pre-determined distances, such as the first amount/distance and second amount/distance described above.

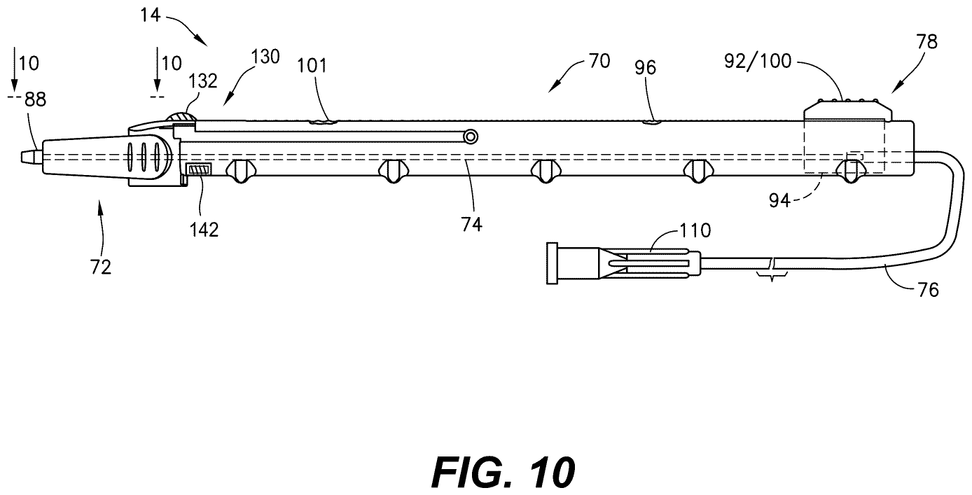

According to another aspect of the disclosure, and as shown in FIGS. 10-13, the fluid transfer device 14 may be further configured to enable an incremental advancement of the flexible tube 74 as it reaches its second position, to enable precise positioning of the distal end 104 of the flexible tube 74 at a number of locations within the vein—i.e., to be incrementally moved distally out past the distal tip 116 of the catheter 24 by a number of pre-determined distances. According to embodiments, a translating mechanism 130 is provided in fluid transfer device 14 that functions similar to a mechanical pencil-like actuator that adjusts the positioning of the flexible tube 74 in an incremental manner. The translating mechanism 130 may be provided at the distal end 82 of the housing 70, such as in/at the coupler 84 of housing 70 and in/at the coupling device 72 (e.g., within threaded connection 91 and blunted cannula 88).

According to an exemplary embodiment, the translating mechanism 130 generally comprises an actuator button 132 that extends up/out through an outer surface of the housing 70 at the distal end 82 thereof, a chuck ring 134 positioned within housing 70 and/or coupling device 72 and about flexible tube 74, a clamping chuck 136 positioned within housing 70 and/or coupling device 72 and movable relative to the chuck ring 134, and a spring 138 positioned about a portion of the clamping chuck 136 and within housing 70. The clamping chuck 136 is operably connected to the actuator button 132, such that depression of the actuator button 132 causes a corresponding distal movement of the clamping chuck 136.

In operation of the fluid transfer device 14, the fluid transfer device 14 may initially be operated as generally shown and described in FIGS. 4 and 5, with the advancement member 78 being advanced distally along housing 70 to move the flexible tube 74 from the first position toward the second position. As the flexible tube 74 approaches the second position, such as with the distal end 104 of the flexible tube 74 being brought even with the distal tip 116 of the catheter 24 (or slightly distally past the distal tip 116), the translating mechanism 130 of the fluid transfer device 14 may then be utilized to move the flexible tube 74 further distally in small and precise pre-determined increments. In some embodiments, the translating mechanism 130 is used during infusion of a caustic medication, to reposition the distal end 104 of the flexible tube 74 within the vein and thereby de-localize, which lessens the risk of tissue damage in the tunica intima layer.

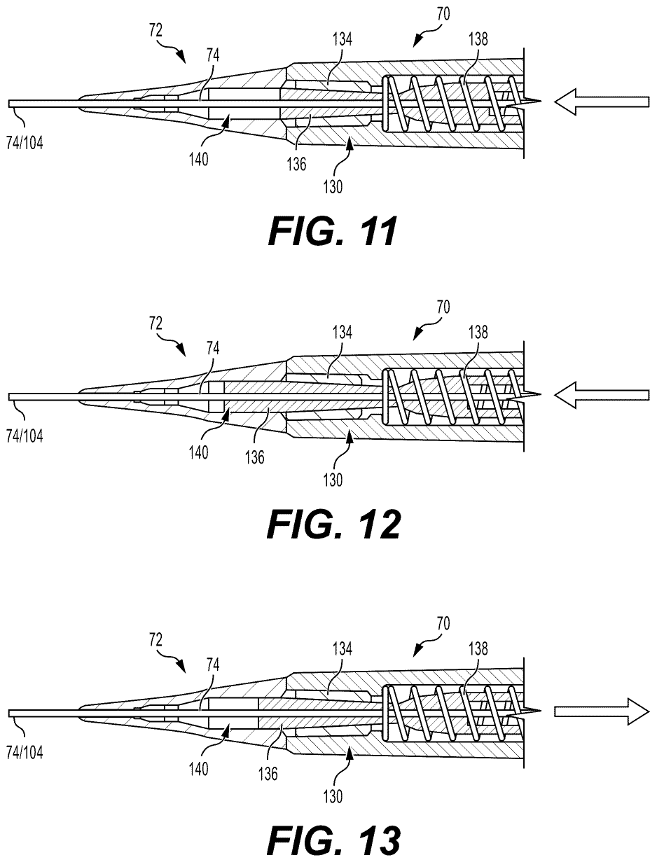

In order to operate the translating mechanism 130, a user depresses the actuator button 132, with depression of the button 132 transferring an actuating force and motion to the clamping chuck 136 that causes the clamping chuck 136 to distally advance relative to the chuck ring 134 and to clamp or pinch down onto the flexible tube 74, such that the flexible tube 74 is gripped thereby. The clamping chuck 136 moves distally within an inner lumen or cavity 140 of the coupling device 72 from an initial position (FIG. 11) to an actuated position (FIG. 12), until the clamping chuck 136 reaches a proximal end of the cavity 140, with this movement of the clamping chuck 136 causing a corresponding distal movement of the flexible tube 74. According to embodiments, this movement of the clamping chuck 136 and flexible tube 74 is in a known increment (e.g., 1 mm increment), such that each depression of the actuator button 132 provides a distal advancement of the flexible tube 74 by a known length/distance. After the actuator button 132 is released by the user, the actuating force/motion applied to the clamping chuck 136 is ended, such that the clamping chuck 136 is caused to release the flexible tube 74 (i.e., the pinching force applied by the clamping chuck 136 onto flexible tube 74 ends) and the clamping chuck 136 moves back proximally within the cavity of coupling device 72 under the biasing force of spring 138 (FIG. 13). Thus, clamping chuck 136 is returned to its initial/default position and is ready for a subsequent actuation upon another depression of button 132.

In some embodiments, the translating mechanism 130 further comprises a return spring 142 (see FIG. 10) positioned within the housing 70 that functions to retract the flexible tube 74 upon the flexible tube 74 reaching its full distal position (i.e., the second position). The return spring 142 is positioned within the inner volume 83 of housing 70 at a location where the return spring 142 contacts the second portion 94 of advancement member 78 when the advancement member 78 is moved to its distal-most position on housing 70. That is, with translating mechanism 130 being used to incrementally advance the flexible tube 74 to its full distal position (under action of the clamping chuck 136), the advancement member 78 is also caused to move to its distal-most position on housing 70, as the proximal end of flexible tube 74 is secured to the second portion 94 of advancement member 78. Upon the advancement member 78 being moved to its distal-most position on housing 70, the second portion 94 of advancement member 78 presses against the return spring 142 and causes the return spring 142 to compress. As the return spring 142 becomes fully compressed, it applies a proximally-directed force to the advancement member 78 that causes the advancement member 78 and flexible tube 74 to retract back proximally to a position where the distal end 104 of the flexible tube 74 is approximately even with the distal tip 116 of the catheter 24—i.e., an initial position before use/actuation of the translating mechanism 130.

While the translating mechanism 130 is described above as including only a single actuator button 132, it is recognized that the translating mechanism 130 could include a pair of actuator buttons—with one button provided for distally advancing the flexible tube 74 and another button provided for proximally retracting the flexible tube 74.

In some embodiments, operation of the translating mechanism 130 may be automated or semi-automated, such that incremental advancement of the flexible tube 74 can be performed according to a pre-determined schedule. As one example, an automated actuation device could be provided that includes a camshaft positioned adjacent to actuator button 132. As the camshaft is rotated at a predetermined rate/speed, the camshaft is caused to periodically depress the button 132, so as to cause the flexible tube 74 to be distally advanced via actuation of the clamping chuck 136, as described in detail above.

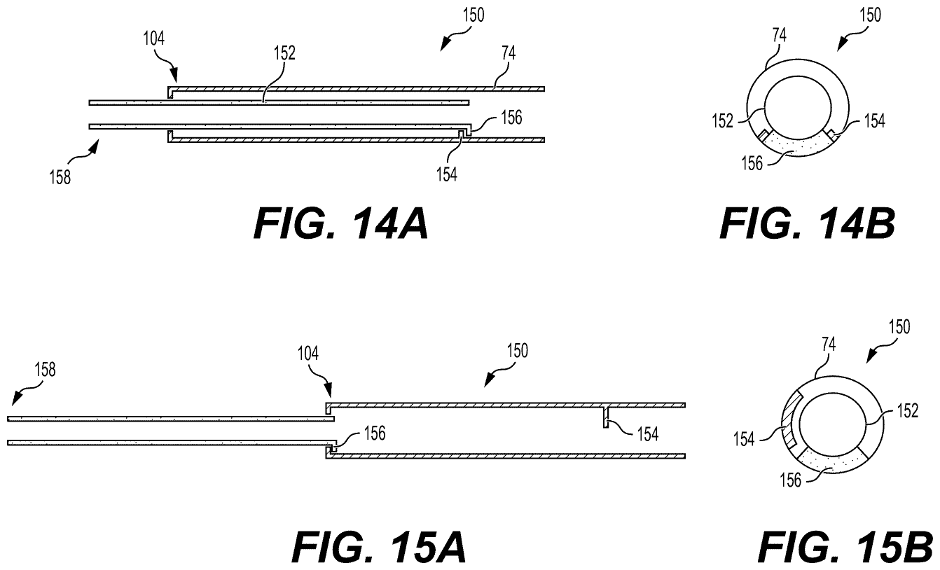

According to another aspect of the disclosure, and as shown in FIGS. 14A and 14B and FIGS. 15A and 15B, the fluid transfer device 14 may be further configured to include a telescoping flexible tube 150 that may be extended to reposition a distal end or tip of the flexible tube as it reaches its second position. More specifically, the telescoping flexible tube 150 may comprise the “main” flexible tube 74 and an inner telescoping tube portion 152 that—in an initial position—is nested substantially (or completely) within the main flexible tube 74, and that—in an extended position—extends out further past the distal end 104 of the flexible tube 74.

According to embodiments of the disclosure, each of the inner telescoping tube portion 152 and the main flexible tube 74 comprise a feature thereon that allows for selective engagement and disengagement of the inner telescoping tube portion 152 from the main flexible tube 74, so as to prevent or allow extension of the inner telescoping tube portion 152 out from the flexible tube 74. In an exemplary embodiment, a semi-circular lip 154 may be formed on an inner surface of the main flexible tube 74 that extends radially inward, while the inner telescoping tube portion 152 includes a corresponding semi-circular lip 156 formed on an outer surface thereof that extends radially outward. When the lip 154 of main flexible tube 74 is circumferentially aligned with the lip 156 of inner telescoping tube portion 152, as shown in FIGS. 14A and 14B, the inner telescoping tube portion 152 is locked in-place relative to the main flexible tube 74 such that it remains nested within the main flexible tube 74. When the lip 154 of main flexible tube 74 is circumferentially mis-aligned with the lip 156 of inner telescoping tube portion 152, as shown in FIGS. 15A and 15B, such as upon a rotational force being applied to the main flexible tube 74, the inner telescoping tube portion 152 is released from the main flexible tube 74 such that it is allowed to extend out from the main flexible tube 74—with a distal tip 158 of inner telescoping tube portion 152 extending distally out past the distal end 104 of flexible tube 74.

In operation of the fluid transfer device 14, the fluid transfer device 14 may initially be operated as generally shown and described in FIGS. 4 and 5, with the advancement member 78 being advanced distally along housing 70 to move the flexible tube 74 from the first position toward the second position. As the flexible tube 74 approaches the second position, such as with the distal end 104 of the flexible tube 74 being brought even with the distal tip 116 of the catheter 24 or slightly out distally past the distal tip 116 of the catheter 24, a user may apply a rotational or twisting force to the housing 70 of the fluid transfer device 14, with this rotational force being transferred to the main flexible tube 74 (due to securing of the flexible tube 74 in the second portion 94 of advancement member 78) and causing the main flexible tube 74 to rotate (e.g., rotate 90 degrees). The rotation of the main flexible tube 74 causes the lip 154 of main flexible tube 74 to become mis-aligned with the lip 156 of inner telescoping tube portion 152, such that the inner telescoping tube portion 152 is released from the main flexible tube 74 and allowed to move distally out from the main flexible tube 74 (FIGS. 15A and 15B). Upon initiation of a fluid infusion of the caustic medication to the patient—with the caustic medication flowing through the main flexible tube 74—the inner telescoping tube portion 152 is caused to extend distally out from the main flexible tube 74 responsive to friction forces exerted on the inner telescoping tube portion 152 by the infused caustic medication. That is, as the caustic medication flows distally through the main flexible tube 74 and inner telescoping tube portion 152, friction created by this fluid flow applies a distally directed force to the inner telescoping tube portion 152 that causes the inner telescoping tube portion 152 to move distally relative to the main flexible tube 74. Accordingly, as caustic medication is infused to the patient, the distal tip 158 of the inner telescoping tube portion 152 may be repositioned within the vein to de-localize administering of the caustic medication—with caustic medication being delivered to a first location within the vein when the inner telescoping tube portion 152 is nested within the main flexible tube 74 and caustic medication being delivered to a second location within the vein when the inner telescoping tube portion 152 is extended out from the main flexible tube 74.

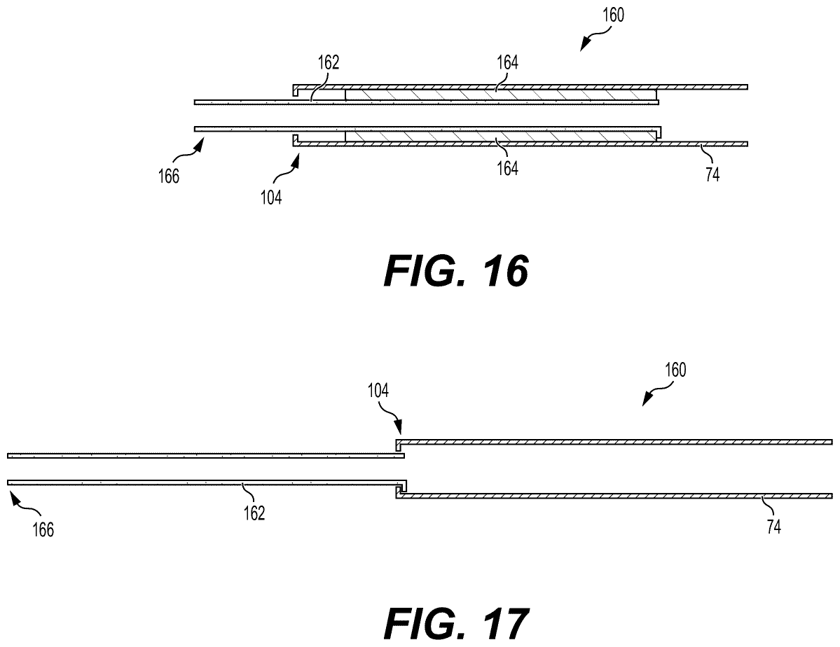

According to another aspect of the disclosure, and as shown in FIGS. 16 and 17, the fluid transfer device 14 includes another embodiment of a telescoping flexible tube 160 that may be extended to reposition a distal end or tip of the telescoping flexible tube 160 as it reaches its second position. Similar to the embodiment of FIGS. 14A and 14B and FIGS. 15A and 15B, the telescoping flexible tube 160 may comprise the “main” flexible tube 74 and an inner telescoping tube portion 162 that—in an initial position—is nested substantially (or completely) within the main flexible tube 74, and that—in an extended position—extends out further past the distal end 104 of the flexible tube 74. In the embodiment of FIGS. 16 and 17, the inner telescoping tube portion 162 is initially retained in-place within the main flexible tube 74 by an adhesive material 164 applied between an outer surface of the inner telescoping tube portion 162 and an inner surface of the main flexible tube 74 (FIG. 16). The adhesive material 164 is specifically selected so that it dissolves upon contact with the caustic medication that is infused to the patient.

In operation of the fluid transfer device 14, the fluid transfer device 14 may initially be operated as generally shown and described in FIGS. 4 and 5, with the advancement member 78 being advanced distally along housing 70 to move the flexible tube 74 from the first position toward the second position. That is, the advancement member 78 is moved distally along housing 70 such that the distal end 104 of the flexible tube 74 is brought even with the distal tip 116 of the catheter 24 or slightly out distally past the distal tip 116 of the catheter 24. With the flexible tube 74 in this position, a fluid infusion of the caustic medication to the patient may be initiated-with the caustic medication flowing through the main flexible tube 74 and through the inner telescoping tube portion 162 while the inner telescoping tube portion 162 is in its initial/nested position. As the caustic medication comes into contact with the adhesive 164 that is applied between the main flexible tube 74 and the inner telescoping tube portion 162 (at the distal end of flexible tube 74), the caustic medication dissolves the adhesive 164 and the adhesive bonds between the main flexible tube 74 and the inner telescoping tube portion 162. Upon the adhesive 164 dissolving, the inner telescoping tube portion 162 is no longer secured to the main flexible tube 74, and the flow of caustic medication distally through the main flexible tube 74 and inner telescoping tube portion 162 creates friction that applies a distally directed force to the inner telescoping tube portion 162 that causes the inner telescoping tube portion 162 to move distally relative to the main flexible tube 74 (FIG. 17). Accordingly, as caustic medication is infused to the patient, a distal tip 166 of the inner telescoping tube portion 162 may be repositioned within the vein to de-localize administering of the caustic medication-with caustic medication being delivered to a first location within the vein when the inner telescoping tube portion 162 is nested within the main flexible tube 74 and caustic medication being delivered to a second location within the vein when the inner telescoping tube portion 162 is extended out from the main flexible tube 74.

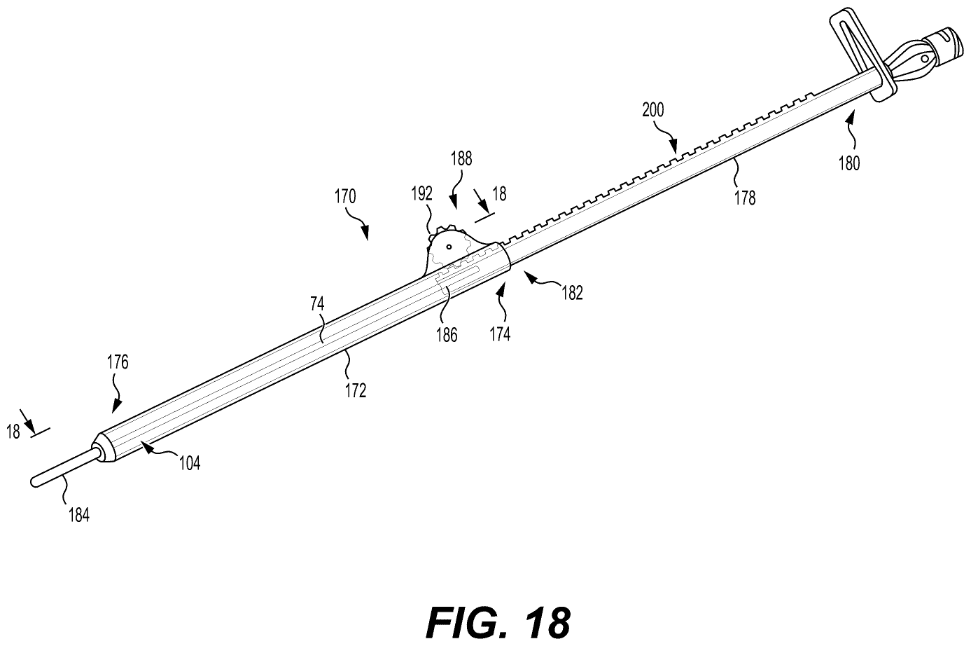

Referring now to FIGS. 18-22, a fluid transfer device 170 is shown according to another aspect of the disclosure, with the fluid transfer device 170 configured and/or controlled during operation to vary/adjust the positioning of the distal end 104 of flexible tube 74 during infusion of the caustic medication, either manually or semi-automatically (e.g., using an externally-controlled motorized mechanism), in order to optimize delivery of the caustic medication and maintain vein health. That is, the flexible tube 74 may be pulled/pushed relative to the catheter 24 (FIG. 1) throughout the duration of a caustic medication infusion/therapy, such that the distal end 104 of the flexible tube 74 may be displaced/shifted within the vein and the administering of the caustic medication may be de-localized, which lessens the risk of tissue damage in the tunica intima layer.

The fluid transfer device 170 includes an outer housing 172 having a proximal end 174 and a distal end 176, and an inner housing 178 slidably received within outer housing 172 and having a proximal end 180 and a distal end 182. In non-limiting embodiments, inner housing 178 and outer housing 172 are in a telescoping relationship, such that inner housing 178 may be slidably received entirely, or almost entirely, within outer housing 172.

Flexible tube 74 is received within outer housing 172 and may be advanced and/or retracted relative to outer housing 172 by displacement of the inner housing 178 relative to the outer housing 172. In non-limiting embodiments, flexible tube 74 may be advanced from a first position in which distal end 104 of flexible tube 74 is within fluid transfer device 170, for example within outer housing 172 and/or a coupler 184 (e.g., a blunted cannula) that secures the fluid transfer device 170 to the proximal coupler 30 of a catheter assembly 12, as shown in FIG. 1, to a second position, in which a distal end 104 of flexible tube 74 is positioned distally of coupler 184 and, in embodiments in which fluid transfer device 170 is coupled to catheter assembly 10, optionally distally of catheter 22.

Flexible tube 74 may be joined at a fitting 186 to inner housing 178. According to non-limiting embodiments, flexible tube 74 may be joined at fitting 186 to a separate fluid tube 166 that passes through inner housing 178 and is coupled to connector 170. In other non-limiting embodiments, flexible tube 74 may be joined at fitting 186 to inner housing 178, with inner housing 178 itself forming a flexible tube in fluid communication with connector 170.

According to embodiments, movement of inner housing 178 and flexible tube 74 relative to outer housing 172, including distal movement and optionally proximal movement thereof, is effected by way of an advancement member 188 that is provided on the outer housing 172. In operation of the fluid transfer device 170, an operator actuates (e.g., rotates) the advancement member 188, which in turn causes a linear movement of the inner housing 178 and flexible tube 74, such as advancement or retraction of the inner housing 178 and flexible tube 74. In some embodiments, the advancement member 188 may be positioned on outer housing 172 at the proximal end 174 thereof, while in other embodiments the advancement member 188 may be positioned on outer housing 172 at the distal end 176 thereof. Outer housing 172 includes a compartment 190 formed therein configured to at least partially house the advancement member 188.

According to embodiments of the disclosure, advancement member 188 includes an advancement wheel 192 mounted on an axle 193 that maintains the advancement wheel 192 within compartment 190 and allows the advancement wheel 192 to rotate in a first direction 194 and a second direction 196. An upper portion of the advancement wheel 192 extends upwardly from compartment 190 and outside outer housing 172, so as to be engageable by an operator, while a bottom portion of the advancement wheel 192 is positioned so as to be engaged with the inner housing 178, as explained in greater detail below.

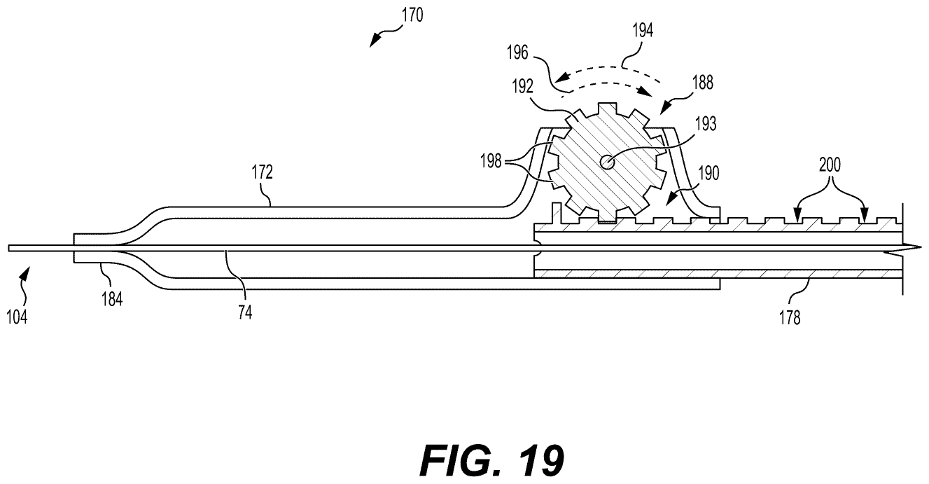

As shown in FIG. 19, the advancement wheel 192 includes teeth 198 thereon that are configured to engage with a grooved track 200 formed along a top surface of the inner housing 178. With engagement of the teeth 198 and grooved track 200, the advancement wheel 192 and inner housing 178 thus form a rack and pinion type engagement that functions to urge the inner housing 178 (and flexible tube 74) toward the distal end 176 of the outer housing 172 or back toward the proximal end 174 of the outer housing 172, depending on which direction the advancement wheel 192 is rotated. In some embodiments, rotation of the advancement wheel 192 and interaction between the teeth 198 and grooved track 200 provide an audible and/or tactile feedback to a user—with each engagement of a tooth 198 and grooved track 200 resulting from rotation of the advancement wheel 192 causing an audible click or a tactile sensation for the user.

In operation of the fluid transfer device 170, rotation of the advancement wheel 192 in a second direction 196 (i.e., toward the proximal end 174 of the outer housing 172) causes the rack and pinion type engagement of advancement wheel 192 and inner housing 178 to advance the inner housing 178 distally forward. The inner housing 178 thus moves/slides toward the distal end 176 of the outer housing 172, and this, in turn, causes the flexible tube 74 to advance distally, such that the distal end 104 of the flexible tube 74 may be extended out from outer housing 172 and coupler 184 and into catheter assembly 12. Conversely, rotation of the advancement member 188 in a first direction 194 (i.e., toward the distal end 176 of the outer housing 172), causes the rack and pinion type engagement of advancement wheel 192 and inner housing 178 to retract the inner housing 178 proximally backward. The inner housing 178 thus moves/slides toward the proximal end 174 of the outer housing 172, and this, in turn, causes the flexible tube 74 to retract proximally, such that the distal end 104 of the flexible tube 74 may be retracted back into outer housing 172.

According to aspects of the disclosure, rotation of the advancement wheel 192 in the second direction 196 (i.e., toward the proximal end 174 of the outer housing 172) causes the flexible tube 74 to advance distally, with each engagement of a tooth 198 and the grooved track 200 resulting from a slight rotation of the advancement wheel 192 moving the flexible tube 74 in the distal direction by a known finite increment. The advancement wheel 192 may be rotated to distally advance (or proximally retract) the flexible tube 74 while the caustic medication is being infused to the patient, with such rotation of the advancement wheel 192 precisely positioning the distal end 104 of flexible tube 74 relative to the distal tip 116 of the catheter 24 during infusion of the caustic medication, in order to delocalize delivery of the caustic medication inside the vein.

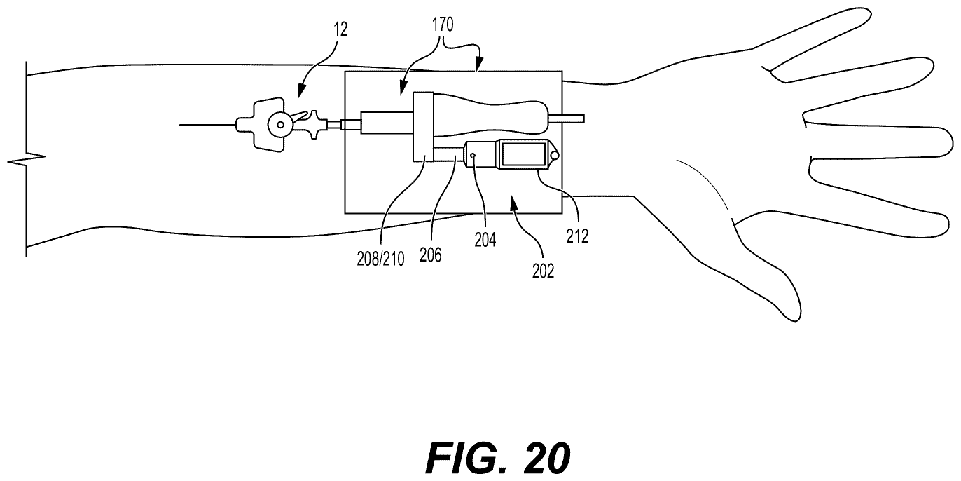

In some embodiments, and as shown in FIGS. 20-22, actuation of the advancement wheel 192 may be performed in an automated or semi-automated fashion via an externally-controlled, motorized translating mechanism 202 (hereafter, “translating mechanism 202”) that is operable with the fluid transfer device 170. The translating mechanism 202 may be secured to a patient (alone or along with fluid transfer device 170) at a location adjacent to an insertion site of the catheter 24, such as via a stabilization device secured to the skin of the patient. As explained in further detail below, the translating mechanism 202 is further attached/coupled to the fluid transfer device 170, i.e., to advancement wheel 192, such that the translating mechanism 202 may operate to cause a semi-automatic movement of the flexible tube 74 during infusion of the caustic medication.

According to one embodiment, and as shown in FIG. 20, the translating mechanism 202 may be configured similar to the translating mechanism 202 previously described for FIGS. 7-9, with the translating mechanism 202 configured as a linear actuator that includes a motor 204, a ball screw 206, and a nut or coupler 208. The motor 204 is operable to cause rotation of the ball screw 206, with rotation of the ball screw 206 in turn causing a linear motion of the coupler 208 (which is positioned about the ball screw 206). A linkage arrangement 210 is provided that connects the coupler 208 to the advancement wheel 192, with the linkage arrangement 210 configured to translate a linear motion of the coupler 208 into a rotational motion that is applied to the advancement wheel 192—thereby causing the advancement wheel 192 to rotate, which also moves the flexible tube 74. In some embodiments, translating mechanism 202 may control movement of the advancement wheel 192 to provide for a full range of positioning of the flexible tube 74 between the first position and the second position thereof, as previously described. The translating mechanism 202 is operable to precisely control positioning of the distal end 104 of flexible tube 74 relative to the distal tip 116 of the catheter 24 during infusion of the caustic medication, in order to delocalize delivery of the caustic medication inside the vein.

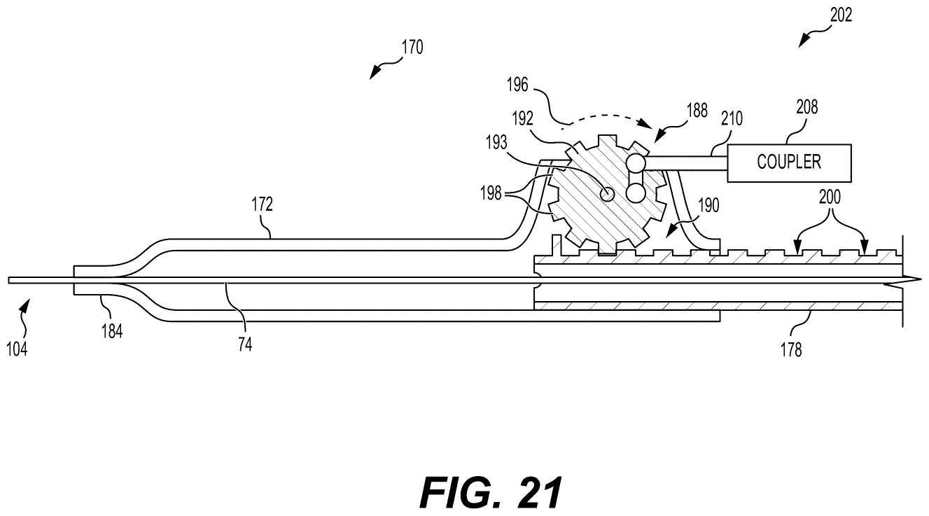

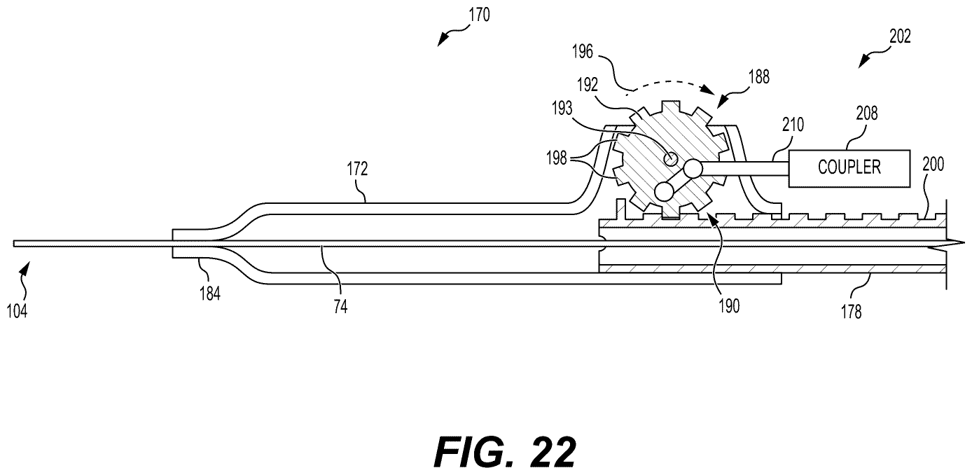

Operation of the translating mechanism 202 to cause movement of the advancement member 78 and flexible tube 74 is shown in FIGS. 21 and 22. As shown first in FIG. 21, translating mechanism 202 may be controlled to perform a first actuation, where linear motor 204 causes ball screw 206 to rotate and cause a corresponding linear movement of coupler 208—with the linear movement of coupler 208 causing a rotational movement of linkage arrangement 210 that is transferred to the advancement wheel 192, so as to rotate the advancement wheel 192. As the advancement wheel 192 is rotated in the second direction 196 (i.e., toward the proximal end 174 of the outer housing 172), the rack and pinion type engagement of advancement wheel 192 and inner housing 178 advances the inner housing 178 distally forward, which in turn distally advances the flexible tube 74 within catheter 24—with FIG. 21 showing movement of the flexible tube 74 that repositions the distal end 104 from a location even with the distal tip 116 of catheter 24 to a first location where the distal end 104 extends distally past the distal tip 116 by a first amount/distance. Movement of the distal end 104 flexible tube 74 to the first position may be performed while infusion of the caustic fluid is occurring.

As shown first in FIG. 22, translating mechanism 202 may be controlled to perform a second actuation, where linear motor 204 causes ball screw 206 to rotate and cause a corresponding linear movement of coupler 208—with the linear movement of coupler 208 causing a rotational movement of linkage arrangement 210 that is transferred to the advancement wheel 192, so as to rotate the advancement wheel 192. As the advancement wheel 192 is rotated further in the second direction 196, the rack and pinion type engagement of advancement wheel 192 and inner housing 178 advances the inner housing 178 distally forward, which in turn distally advances the flexible tube 74 within catheter 24—with FIG. 22 showing movement of the flexible tube 74 that repositions the distal end 104 from a first location that is distally past the distal tip 116 by a first amount/distance to a second location where the distal end 104 extends distally past the distal tip 116 by a second amount/distance. Again, movement of the distal end 104 flexible tube 74 to the second position may be performed while infusion of the caustic fluid is occurring. In this manner, administering of the caustic medication is de-localized, which lessens the risk of tissue damage in the tunica intima layer.

According to embodiments, the translating mechanism 202 may comprise a battery-operated, re-usable device, that may be selectively attached and detached from the fluid transfer device 170. The motor 204 of translating mechanism 202 may be controlled according to a set program, which may be stored on a control module 212 associated with the motor 204, with the program configured to operate the motor 204 to cause the distal end 104 of the flexible tube 74 to be moved intravenously to a number of pre-determined locations within the vein—i.e., moved distally out past the distal tip 116 of the catheter 24 by a number of pre-determined distances, such as the first amount/distance and second amount/distance described above.

Beneficially, embodiments of the disclosure provide a system and method for infusion of caustic medications into an indwelling PIVC, while reducing the harmful effects of caustic medications on the vein wall. The fluid transfer device enables variation in the location of intravenous administration of the caustic medication, with such distributed delivery lessening the risk of tissue damage to the vein. An externally-controlled, motorized translating mechanism may be utilized with the fluid transfer device that provides for semi-automatic control in adjusting the positioning of the distal end of flexible tube within the vein during infusion of the caustic medication.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A system for intravenous delivery of a caustic medication to a patient, the system comprising:
a catheter assembly including a catheter adapter and a catheter positionable intravenously in the patient;
a fluid source containing a caustic medication; and
a fluid transfer device coupleable to the fluid source and to the catheter assembly and operable to administer the caustic medication to the patient, the fluid transfer device comprising:
a flexible tube configured to be inserted within the catheter, the flexible tube in fluid communication with the fluid source;
a housing having a proximal end and a distal end and defining an inner volume configured to movably receive the flexible tube;
a coupling device positioned at the distal end of the housing and configured to couple the housing to the catheter assembly; and
an advancement member configured to move relative to the housing to move the flexible tube between a first position, in which a distal end of the flexible tube is disposed within the housing or within the coupling device, and a second position, in which the distal end of the flexible tube is disposed within the catheter or past a distal tip of the catheter, with the flexible tube able to transfer the caustic medication from the fluid source to the patient when in the second position;
wherein, with the flexible tube at the second position, the fluid transfer device is configured to further incrementally advance the flexible tube distally out past the distal tip of the catheter by a finite distance, to reposition the distal end of the flexible tube within a vein of the patient during administering of the caustic medication.

2. The system of claim 1, wherein the advancement member comprises a first portion and a second portion, with the first portion movably disposed along an upper surface of the housing and the second portion movably disposed within an inner volume of the housing, with the advancement member movable distally along the housing to move the flexible tube from the first position to the second position.

3. The system of claim 2, wherein the fluid transfer device further comprises a linear actuator configured to move the advancement member distally along the housing, the linear actuator including a motor, a ball screw, and a coupler, with the motor operating to cause rotation of the ball screw and rotation of the ball screw, in turn, causing a linear motion of the coupler, and wherein portion of the coupler engages the first portion of the advancement member, such that the linear motion of the coupler is transferred to the advancement member.

4. The system of claim 3, wherein the linear actuator comprises a control module having a program stored thereon operates the motor to cause the distal end of the flexible tube to be incrementally advanced distally out past the distal tip of the catheter by a finite distance for each actuation of the motor, to reposition the distal end of the flexible tube within the vein of the patient.

5. The system of claim 1, wherein the housing comprises an outer housing, and wherein the fluid transfer device further comprises an inner housing slidably received within the outer housing, the inner housing including a grooved track formed along a top surface thereof, with a proximal end of the flexible tube secured to the inner housing; and
wherein the advancement member comprises an advancement wheel secured to the outer housing that is rotatable in a first direction and a second direction, where rotation in the second direction causes the flexible tube to move toward the second position, with the advancement wheel comprising teeth thereon that interact with the grooved track to advance the inner housing and the flexible tube distally when the advancement wheel is rotated in the second direction.

6. The system of claim 5, wherein rotation of the advancement wheel in the second direction causes the flexible tube to advance distally, with each engagement of the teeth of the advancement wheel and the grooved track resulting from rotation of the advancement wheel moving the flexible tube in the distal direction by a known finite increment, to reposition the distal end of the flexible tube within the vein of the patient.

7. The system of claim 5, wherein the fluid transfer device further comprises an actuator configured to rotate the advancement wheel in the first direction and the second direction, the actuator including a motor, a ball screw, a coupler, and a linkage arrangement, with the linkage arrangement connecting the coupler to the advancement wheel; and
wherein the motor operates to cause rotation of the ball screw and rotation of the ball screw, in turn, causing a linear motion of the coupler, and the linkage arrangement translating the linear motion of the coupler into a rotational motion that is applied to the advancement wheel to cause rotation thereof in the first direction or the second direction.

8. The system of claim 7, wherein the actuator comprises a control module having a program stored thereon operates the motor to cause the distal end of the flexible tube to be incrementally advanced distally out past the distal tip of the catheter by a finite distance for each actuation of the motor, to reposition the distal end of the flexible tube within the vein of the patient.

9. The system of claim 1, wherein the flexible tube comprises a telescoping flexible tube comprising a main flexible tube and an inner telescoping tube portion positioned at a distal end of the main flexible tube, the inner telescoping tube portion movable between an initial position where the inner telescoping tube portion is nested substantially or completely within the main flexible tube and an extended position where the inner telescoping tube portion extends out further past the distal end of the main flexible tube.

10. The system of claim 9, wherein the main flexible tube comprises a first lip formed on an inner surface thereof and the inner telescoping tube portion comprises a second lip formed on an outer surface thereof, and wherein the inner telescoping tube portion is retained in the initial position when the first lip and the second lip are aligned and engaged and the inner telescoping tube portion is released from the main flexible tube when the first lip and the second lip are misaligned and disengaged, thereby allowing the inner telescoping tube portion to move to the extended position.

11. The system of claim 10, wherein the first lip and the second lip are moved from an aligned and engaged arrangement to a misaligned and disengaged arrangement responsive to rotation of the housing of the fluid transfer device.

12. The system of claim 10, wherein with the inner telescoping tube portion released from the main flexible tube, the inner telescoping tube portion moves from the initial position to the extended position responsive to friction forces exerted on the inner telescoping tube portion by a flow of the caustic medication as it is administered to the patient.

13. The system of claim 9, wherein the telescoping flexible tube further comprises an adhesive applied between an inner surface of the main flexible tube and an outer surface of the inner telescoping tube portion, the adhesive retaining the inner telescoping tube portion in the initial position.

14. The system of claim 13, wherein the adhesive is configured to dissolve upon contact with the caustic medication that is infused to the patient, to release the inner telescoping tube portion from the main flexible tube, thereby allowing the inner telescoping tube portion to move to the extended position; and
wherein with the inner telescoping tube portion released from the main flexible tube, the inner telescoping tube portion moves from the initial position to the extended position responsive to friction forces exerted on the inner telescoping tube portion by a flow of the caustic medication as it is administered to the patient.

15. The system of claim 1, wherein the fluid transfer device further comprises a translating mechanism comprising:
an actuator button that extends up through an outer surface of the housing at the distal end thereof;
a chuck ring positioned within the housing and/or the coupling device and about the flexible tube;
a clamping chuck positioned within the coupling device and movable relative to the chuck ring, with the clamping chuck operably connected to the actuator button; and
a spring positioned about a portion of the clamping chuck and within the housing;
wherein depression of the actuator button transfers an actuating force and motion to the clamping chuck that causes the clamping chuck to distally advance relative to the chuck ring and to pinch down onto the flexible tube, such that the flexible tube is gripped thereby, with the distal advancing of the clamping chuck causing a corresponding incremental distal movement of the flexible tube by a finite distance, to reposition the distal end of the flexible tube within the vein of the patient.

16. The system of claim 15, wherein the clamping chuck moves distally when the actuator is depressed until reaching a proximal end of a cavity within the coupling device, and wherein the clamping chuck releases the flexible tube and moves back proximally within the coupling device upon release of the actuator button, with a biasing force of the spring causing the proximal movement of the clamping chuck back to an initial position.

17. The system of claim 15, wherein the translating mechanism further comprises a return spring positioned within the housing and configured to retract the flexible tube upon the flexible tube reaching a full distal position, with the return spring compressed by the advancement member when the advancement member is moved to a distal-most position on the housing and applying a proximally-directed force to the advancement member that causes the advancement member and the flexible tube to retract back proximally.

18. A method of intravenously delivering a caustic medication to a patient using the system of claim 1, the method comprising:
connecting the fluid transfer device to the catheter assembly with the distal tip of the indwelling intravenously in the patient;
connecting the fluid source to the fluid transfer device such that the fluid source is fluidly connected with the flexible tube;
actuating the advancement member to move the flexible tube from the first position to the second position; and
with the flexible tube in the second position, operating the fluid source to administer the caustic medication to the patient, via the flexible tube;
wherein, during administration of the caustic medication to the patient, the fluid transfer device is operated to move the distal end of the flexible tube to a plurality of positions that are all distal to the distal tip of the catheter, to vary an intravenous location within the patient at which the caustic medication is administered.

19. The method of claim 18, wherein operating the fluid transfer device during administration of the caustic medication to move the distal end of the flexible tube comprises operating a translating mechanism of the fluid transfer device to move the distal end of the flexible tube in a semi-automatic manner.

20. The method of claim 18, wherein operating the fluid transfer device during administration of the caustic medication to move the distal end of the flexible tube comprises extending a nested inner telescoping tube portion out from the flexible tube, to reposition a distal-most tip of the inner telescoping tube portion intravenously within the patient.
